(19) United States
(12) Patent Application Publication
(10) Pub. No.: US 2006/0083710 A1
(43) Pub. Date: Apr. 20, 2006

(54) Process for making antimicrobial polymer articles

(76) Inventors: Melissa C. Joerger, Newark, DE (US); Subramaniam Sabesan, Newark, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/252,700

(22) Filed: Oct. 18, 2005

Related U.S. Application Data
(60) Provisional application No. 60/619,755, filed on Oct. 18, 2004.

(51) Int. Cl. A61L 9/01; B32B 27/04

(52) U.S. Cl. 424/76.1; 442/128

(57) ABSTRACT

This invention relates to a process for making articles antimicrobial and odor inhibiting, which comprises using vacuum deposition and electron beam techniques to graft amino-reactive functional groups onto polymeric material which the article comprises, followed by contacting the polymeric material with a chitosan solution.

### FIELD OF THE INVENTION

This invention is directed to a process for rendering a polymer article antimicrobial comprising using vacuum deposition and electron beam techniques to graft amino-reactive functional groups onto an article, followed by contacting with a chitosan solution.

### TECHNICAL BACKGROUND OF THE INVENTION

As evidenced by the presence in the market of numerous materials for eliminating or minimizing human contact with microbes, there is clearly a demand for materials and/or processes that either minimize or kill microbes encountered in the environment. Such materials are useful in areas of food preparation or handling and in areas of personal hygiene, such as bathrooms. Similarly, there is a use for such antibacterial materials in hospitals and nursing homes where people with lowered resistance are especially vulnerable to illness-causing microbes.

There is also growing demand to inhibit odor development in many applications. Humans possess several areas on the body where odors can be produced. We also have the ability to detect thousands of odorants. Among our most notable odor-producing areas are the axillae, genital regions and feet, which produce the largest array of odorants and have been the subject of numerous studies as well as the focus of many consumer products. In addition, odors are produced on the skin of the neck, torso, arms and legs, which contain large numbers of sebaceous as well as eccrine glands and support a population of Staphylococcus (notably S. epidermidis). Some regions of skin contain high concentrations of apocrine or apoeccrine glands, e.g., the axillae, nipples, and ano-genital region. Apocrine secretions from these regions produce distinct and often quite strong odors, which may become malodorous. This results, in part, from the activity of bacteria, which hydrolyze the proteins in these secretions, thereby releasing malodorants (Spielman, A. I., Zeng, X-N., Leyden, J. J. and Preti, G. Proteinaceous precursors of human axillary odor: isolation of two novel odor binding proteins. Experientia, 1995, 51, 4044). Since consumers desire the perception of a scent of freshness in apparel and hygiene products, it is desirable to find practical methods of inhibiting odor development.

In addition, there are packaging applications that can benefit from odor control functionality. Certain foods, including meats, can naturally produce odors that concentrate within a package. When the package is opened, even though the food product is still fresh, the consumer may detect undesirable odors. For example, poultry products are rich in proteins containing disulfide bonds. When poultry products are packaged in barrier or modified atmosphere packaging, the sulfide odors that naturally form cannot dissipate, accumulate and concentrate within the package. Upon opening the package, the consumer detects a strong, unpleasant sulfide smell.

Chitosan is the commonly used name for poly-[1-4]-β-D-glucosamine. Chitosan is chemically derived from chitin which is a poly-[1-4]-β-N-acetyl-D-glucosamine, which, in turn, is derived from the cell walls of fungi, the shells of insects and, especially, crustaceans. Thus, it is inexpensively derived from widely available materials. It is available as an article of commerce from, for example, Biopolymer Engineering, Inc. (St. Paul, Minn.); Biopolymer Technologies, Inc. (Westborough, Mass.); and CarboMer, Inc. (Westborough, Mass.).

Chitosan can be treated with metal salt solutions so that the metal ion forms a complex with the chitosan. Chitosan and chitosan-metal compounds are known to provide antimicrobial activity as bacteriocides and fungicides (see, e.g., T. L. Vigo, “Antimicrobial Polymers and Fibers: Retrospective and Prospective,” in Bioactive Fibers and Polymers, J. V. Edwards and T. L. Vigo, eds., ACS Symposium Series 792, pp. 175-200, American Chemical Society, 2001). Chitosan is also known to impart antiviral activity, though the mechanism is not yet well understood (see, e.g., Chirkov, S. N., Applied Biochemistry and Microbiology (Translation of Prikladnaya Biokhimiya i Mikrobiologiya) (2002), 38(1), 1-8). Additionally, chitosan is known to impart antiodor properties; see, for example, WO 99/061079.

U.S. Pat. No. 4,326,532 discloses preparation of polymeric surfaces for bonding with chitosan by three methods: (1) with oxygen R_{f} plasma discharge; (2) chromic acid oxidation; or (3) R_{f} plasma polymerization of acids on the surface-, even though the only methods exemplified therein are (1) and (3). However, in this patent, chitosan-coated polyethylene articles are prepared only as controls. In a paper co-authored by the inventor (L. K. Lambrecht et al., Trans. Am. Soc. Artif. Intern. Organs, Vol. XXVII, 380-385, 1981), on transient thrombus deposition on chitosan-heparin coated polyethylene, polyethylene tubings are primed for chitosan coating by exposure to a chromic acid solution. In both of these references, the chitosan/polyethylene articles are only experimental controls and are not under consideration as useful articles in their own right.

U.S. Pat. No. 5,618,622 discloses a surface-modified fibrous filtration medium, which includes hydrocarbon-polymer fibers having cationic or anionic functional groups on the surfaces thereof, coated with a polyelectrolyte of opposite charge, such as chitosan. There is no mention of antimicrobial properties.

U.S. Pat. No. 6,197,322 discloses polypropylene nonwoven fabric treated with chitosan to reduce odors and promote skin wellness, e.g., in diapers. The chitosan was applied by simple dipping. The chitosan was crosslinked to improved durability. Y. Shin, D. I. Yoo, and K. Min (Journal of Applied Polymer Science, Vo. 74, 2911-2916, 1999; Asian Textile Journal, February 2000, 43-45) applied water-soluble chitosan oligomer as an antimicrobial finishing agent for polypropylene nonwoven fabric. The aqueous solution of chitosan oligomer (weight average molecular weight of 1814) was applied by padding.

In co-pending U.S. Patent Application No. 2003/0091612, which is hereby incorporated by reference, polyolefin articles are treated with an aqueous mixture of chromic acid and sulfuric acid, washed with deionized water, soaked in concentrated nitric acid, and again washed with deionized water before treatment with chitosan solution. While effective antimicrobial articles are made by this method, a simpler, more economical process is desirable. It is also desirable to use more environmentally benign materials than strong oxidizers like chromic acid and sulfuric acid, particularly in large-scale applications.

U.S. Pat. No. 5,932,495 discloses substrates containing triglycerides and/or polyglycosides for enhancement of malodor absorption properties of chitosan, alginates, or synthetic polymers.

There remains a need for an effective, efficient, and environmentally benign process to apply chitosan to polymer surfaces to produce articles that are antimicrobial and which inhibit odor development.

### SUMMARY OF THE INVENTION

Described herein are methods for making articles, which comprise a polymeric material, antimicrobial and/or odor inhibiting as well as the antimicrobial and/or odor inhibiting articles made therefrom.

The described methods comprise the steps of:
a) mixing a graft monomer with a crosslinking agent to produce a blend;
b) feeding the blend into a hot evaporator under vacuum;
c) flash evaporating the blend through a nozzle;
d) recondensing the blend onto polymeric material;
e) exposing the recondensed blend to ultraviolet or electron beam radiation, whereby the recondensed blend and the polymeric material react to form a crosslinked graft copolymer;
f) contacting the crosslinked graft copolymer with a solution comprising a chitosan agent selected from the group consisting of chitosan, chitosan salts, chitosan-metal complexes, and chitosan derivatives;
g) optionally, contacting the crosslinked graft copolymer with a solution containing a metal salt; and
h) drying the crosslinked graft copolymer; wherein the contacted crosslinked graft copolymer is antimicrobial and odor inhibiting.

### DETAILED DESCRIPTION OF THE INVENTION

Applicants specifically incorporate the entire content of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

In the context of this disclosure, a number of terms shall be utilized. As used herein, the term “(meth)acrylate” denotes both acrylate and methacrylate.

As used herein, the term “amino-reactive functional groups” refers to chemical functionalities that readily undergo chemical reaction with an NH₂ group. Examples include positively charged species such as metal ions, anhydrides, carboxylic acids, isocyanates, epoxides, acid chlorides, and enones.

As used herein, the term “polyolefin” refers to olefinic homopolymers and to copolymers of at least one olefin and at least one other comonomer which may or may not be another olefin.

As used herein, the term “polymeric material” refers to material whose surface comprises at least one polymer.

As used herein, the term “grafted” refers to a species that is bound to a polymeric substrate by a chemical bond. The chemical bond includes, but is not limited to ionic and covalent bonds.

As used herein, the term “graft copolymer” refers to a copolymer with one or more side chains connected to the main chain, or “backbone” In a graft copolymer, the distinguishing feature of the side chains is constitutional, i.e., the side chains comprise units derived from at least one species of monomer different from those which supply the units of the main chain. An example, wherein A is a backbone monomer, X is the graft site, and B is the sidechain monomer:

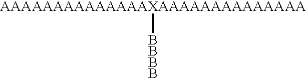

As used herein, the term “crosslinked” refers to a polymer in which adjacent polymer chains are joined at various positions by covalent bonds.

As used herein, the term “crosslinked graft copolymer” refers to a graft copolymer in which pairs of adjacent sidechains are crosslinked. An example, wherein the B—B bond is the crosslink:

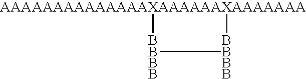

As used herein, the term “nonwoven” refers to a manufactured sheet, web or batt of directionally or randomly orientated fibers, bonded by friction and/or cohesion and/or adhesion. This term excludes paper and products, which are woven, knitted, tufted, stitch-bonded incorporating binding yarns or filaments, or felted by wet-milling, whether or not additionally needled.

As used herein, the term “antimicrobial” as used herein, means bactericidal, fungicidal, and antiviral as is commonly known in the art. By “microbial growth is reduced”, “reduction of bacterial growth” or “sufficient to reduce microbial growth” is meant that a 99.9% kill of the bacteria in 24 hours has been met as measured by the Shake Flask test described below and commonly used to measure antimicrobial functionality, which indicates a minimum requirement of a 3-log reduction in bacterial growth.

As used herein, the term “chitosan agent” as used herein means all chitosan-based moieties, including chitosan, chitosan salt, chitosan-metal complexes, and chitosan derivatives.

As used herein, the tem “odor inhibiting” or “inhibiting odor development” or “odor development inhibiting” means reduction of the perceived intensity of odor and/or increase of perceived pleasantness of odor. The standard method for measuring odors is the olfactory method, which means the odorous source is perceived by a panel of people.

Where a range of numerical values is recited herein, unless otherwise stated, the range is intended to include the endpoints thereof, and all integers and fractions within the range. It is not intended that the scope of the invention be limited to the specific values recited when defining a range.

Described herein are articles that are antimicrobial and/or inhibit the development of odor and a process for providing these.

The process described herein may be applied to articles whose surfaces comprise any of a wide variety of polymers, both naturally occurring and synthetic. Examples of suitable naturally occurring polymers include but are not limited to cotton, wood, flax, shellac, silk, wool, natural rubber, leather, and mixtures thereof. Examples of suitable synthetic polymers include but are not limited to homopolymers, copolymers, mixtures, and blends of polyesters, polyetheresters, polyethers, polyamides, polyimides, polyetherimides, polyacetals, polystyrene, polyphenylene oxide, polyphenylene sulfide, polysulfones, poly(meth)acrylates, liquid crystalline polymers, polyetherketones, fluorine-containing polymers, acrylonitrile-styrene-butadiene resins, styrene-butadiene block copolymers, polycarbonates, cellulose-based polymers (e.g., cellulose, rayon, cellulose acetate), urea formaldehyde resins, polyacrylonitrile, epoxy resins, polyurethanes, melamine-formaldehyde resins, silicones, butyl rubber, polychloroprene, and polyolefins.

Blends of naturally occurring polymers and synthetic polymers are also contemplated. For example, wood pulp (WP)/polyethylene terephthalate (“PET”) blends can contain 1-100% WP and 100-1% PET. Typical WP/PET blends include, for example, 55% pine WP/45% PET (2 oz/yd² [68 g/m²]), 55% cedar WP/45% PET (1.5 oz/yd² [51 g/m²]), 70% rayon/30% PET intimate fiber blend (8 mesh pattern, 2.2 oz/yd² [75 g/m²]), 50% lyocell/50% PET intimate fiber blend (1.8 oz/yd² [61 g/m²]), or 55% cedar WP/45% PET (2 oz/yd² [68 g/m²]).

Examples of suitable polymers for use in the described process include polypropylene, e.g., atactic polypropylene, isotactic polypropylene, syndiotactic polypropylene, biaxially oriented polypropylene (BOPP), metallocene-catalyzed polypropylene; polyethylene, e.g., high density polyethylene (HDPE), low density polyethylene (LDPE), linear low density polyethylene (LLDPE), metallocene-catalyzed polyethylene, very low density polyethylene (VLDPE), ultrahigh molecular weight polyethylene (UHMWPE), high performance polyethylene (HPPE); copolymers of ethylene and propylene; copolymers derived from ethylene or propylene and at least one monomer chosen from propylene, methyl acrylate, ethyl acrylate, n-butyl acrylate, methyl methacrylate, acrylic acid, methacrylic acid and carbon monoxide; and copolymers of olefins with a diolefin, such as a copolymer of ethylene, or of propylene, or of ethylene and other olefins, with: linear aliphatic nonconjugated dienes of at least six carbon atoms (such as 1,4-hexadiene) and other dienes, conjugated or not, such as norbornadiene, dicyclopentadiene, ethylidene norbornene, butadiene, and the like. Other suitable backbone polymers are copolymers of ethylene and tetrafluoroethylene, such as Tefzel® ETFE fluoropolymer resin available from E. I. du Pont de Nemours & Co., Inc. (Wilmington, Del.).

Another type of polymer suitable in the described methods includes a copolymer of an olefin with an acrylic and/or methacrylic acid. Ethylene is particularly useful. An example of a commercially available material is Nucrel® ethylene acid copolymer resin available from E. I. du Pont de Nemours & Co., Inc. (Wilmington, Del.).

Polymer blends comprising olefin homopolymers and/or copolymers may be used as long as the blend, after being grafted in the described methods, meets the requirement that the amino groups of the chitosan agent react with the substrate's surface to form a stable coating with a surface concentration of chitosan sufficient to reduce microbial growth.

Graft monomers suitable for use in the methods described herein include thermally stable unsaturated monomers containing amine-reactive functional groups. Examples of such suitable graft monomers for use in the methods described herein include but are not limited to methacrylic acid, acrylic acid, glycidyl methacrylate, 2-hydroxy ethylacrylate, 2-hydroxy ethyl methacrylate, beta-carboxyethyl acrylate, beta-carboxyethyl methacrylate, diethyl maleate, monoethyl maleate, di-n-butyl maleate, maleic anhydride, maleic acid, fumaric acid, itaconic acid, itaconic anhydride, dodecenyl succinic anhydride, 5-norbornene-2,3-anhydride, and nadic anhydride (3,6-endomethylene-1,2,3,6-tetrahydrophthalic anhydride). Itaconic acid and itaconic anhydride are particularly preferred graft monomers.

In these methods, a crosslinking agent is used along with the graft monomer. Suitable agents are multifunctional chemical compounds capable of reacting with and crosslinking the graft monomer and are readily determined by one of skill in the art. For example, a triacrylate compound is a suitable crosslinking agent for itaconic acid.

The articles described herein have chitosan grafted thereon. Chitosan is the commonly used name for poly-[1-4]-β-D-glucosamine. Chitosan is chemically derived from chitin which is a poly-[1-4]-β-N-acetyl-D-glucosamine which, in turn, is derived from the cell walls of fungi, the shells of insects and, especially, crustaceans. Chitosan that is particularly useful for the articles described herein has a degree of N-deacetylation of greater than 85%, and a molecular weight in the range of about 60,000-200,000 Daltons, and low heavy metal content (less than 20 ppm). Typical particle size of the chitosan is between 60-100 mesh.

Articles prepared by the methods described herein exhibit antimicrobial and odor development inhibiting functionality because microbial growth is reduced as the article is commonly used. This means that a 99.9% kill of the microbes in 24 hours has been met as measured by the Shake Flask test described below and commonly used to measure antimicrobial functionality, which indicates a minimum requirement of a 3-log reduction in microbial growth.

As an optional first step of the methods described herein, the outer surface of the polymeric article is cleaned using techniques or cleaning agents commonly known in the art for the specific polymer that the article comprises. For example, the surface of an article comprising polymeric material can be cleaned with C, to C₆ alcohols, dialkyl formamide and acetamide or with other polar solvents capable of extracting plasticizers. The surface of a cleaned article may then, if necessary, be dried by methods commonly known in the art, for example, by vacuum, ambient air drying, oven drying, and air forced drying. A particularly suitable cleaning method for use in the methods described herein is plasma treatment.

Following the optional surface cleaning step, amino-reactive functional groups are generated on the surface of the article by vacuum surface functionalization, according to the methods of Yialzis and Mikhael of Sigma Technologies International Inc., Tucson, Ariz. (see Yializis, A. & Mikhael, M. G., Vacuum surface functionalization of paper and woven or nonwoven materials. 46^{th} Annual Technical Conference Proceedings—Society of Vacuum Coaters (2003), pp. 553-558; and U.S. Pat. Nos. 6,270,841, 6,447,553, and 6,468,595). In this process as applied to the methods herein, the graft monomer is mixed with a crosslinking agent to produce a blend. This blend is fed into a hot evaporator under vacuum; flash evaporated through a nozzle; recondensed onto the polymeric surface of an article, the surface comprising at least one of the aforementioned polymers; and exposed to ultraviolet or electron beam radiation. The ultraviolet or electron beam radiation initiates various polymerization, grafting, and crosslinking reactions among the graft monomer, crosslinking agent, and polymeric surface to form a crosslinked graft copolymer at the surface of the article. Typically, the surface of the polymeric material is cleaned by plasma treatment before the graft monomer/crosslinking agent blend is condensed onto it. The evaporator temperature ranges from about 70 to 350° C. Suitable vacuum is in the range of about 10⁻¹ to 10⁻⁷ torr. The temperature of the polymeric material onto which the blend is condensed is in the range of about −20 to about +30° C.

The article is then treated with chitosan. The treatment comprises soaking or wetting the article with a solution comprising a chitosan agent. Chitosan agents include all chitosan-based moieties, including chitosan, chitosan salt, chitosan-metal complexes, and chitosan derivatives.

The solution comprising the chitosan agent may be aqueous. However, since chitosan by itself is not soluble in water, the chitosan may be solubilized in a solution. Solubility is obtained by adding the chitosan to a dilute solution of a water-soluble, organic acid selected from the group consisting of mono-, di- and polycarboxylic acids. This allows the chitosan to react with the acid to form a water-soluble salt, herein referred to as “chitosan salt.” “Chitosan-metal complexes” are formed by treating chitosan solution with metal salt solutions. Alternatively, “chitosan derivatives,” including N- and O-carboxyalkyl chitosan, which are water-soluble, can be used directly in water instead of chitosan salt. The chitosan may also be dissolved in special solvents like dimethylacetamide in the presence of lithium chloride, or N-methyl-morpholine-N-oxide. Such solubilized chitosan solutions may be used in the described methods instead of aqueous solutions containing chitosan salt or chitosan derivatives.

Typically, the solution comprising a chitosan agent is an aqueous acetic acid solution, preferably about 0.5% to about 5% aqueous acetic acid. An aqueous solution containing 0.1% to 3% chitosan and 0.5% to 1.0% acetic acid is particularly useful. Equally useful is an aqueous solution containing 2% chitosan and 1.5% aqueous acetic acid solution. More useful is an aqueous solution containing 2% chitosan and 0.75% acetic acid. The time of treatment is typically 30 seconds to 30 minutes. The temperature of the treatment is not critical and is typically in the range of room temperature to 90° C.

After treatment with chitosan, the article may be washed, preferably with deionized water. Optionally, the article may then be dried via techniques known in the art. These include ambient air drying, oven drying, and air forced drying. An inert atmosphere, such as nitrogen, may be provided in place of air. The article may be grafted with chitosan in a batch process or in a continuous process, as described in co-pending U.S. Patent Publication No. 2003/0017194.

Articles prepared by the described methods exhibit an antimicrobial property and are expected to inhibit odor development as well. The treatment with chitosan of polymeric material is expected to result in both an antimicrobial functionality and an odor inhibiting functionality even if only one functionality is measured. This is because the odor inhibiting functionality is believed dependent upon chitosan's ability to reduce the growth of microbes that activate odor development. Thus, the antimicrobial functionality of chitosan is believed to necessarily implicate inhibition of odor development. This suggests that odor inhibition, while a separately measurable functionality, does not result from a necessarily independent and distinct functionality of the chitosan.

This antimicrobial property may be enhanced by an optional treatment with metal salts. Metal salts useful in the methods described herein include, for example, zinc sulfate, copper sulfate, silver nitrate, or other water-soluble zinc, copper, and silver salts or mixtures of these. The metal salts are typically applied by dipping, spraying or padding the article with a dilute (0.1% to 5%) solution of the metal salt in water. Alternatively, the metal salts may be used by preforming the chitosan-metal salt, isolating the product, and redissolving the product in dilute acid, such as aqueous acetic acid. The degree of enhancement depends on the particular metal salt used, its concentration, the time and temperature of exposure, and the specific chitosan treatment, that is, the type of chitosan agent, its concentration, the temperature, and the time of exposure.

Articles made antimicrobial and/or odor inhibiting by the methods described herein may be in the form of or comprise a film, membrane, laminate, knit fabric, woven fabric, nonwoven fabric, fiber, filament, yarn, pellet, coating, or foam. Two examples of nonwoven materials are DuPont™ Tyvek® brand spunbonded olefin and spunlaced DuPont™ Sontara® Technologies fabrics, both available from E.I. du Pont de Nemours & Co., Inc. (Wilmington, Del.). These articles may be prepared by any means known in the art, such as, but not limited to, methods of injection molding, extruding, blow molding, thermoforming, solution casting, film blowing, knitting, weaving, or spinning.

These articles provide multiple uses, since many articles benefit from a reduction in microbial growth and/or inhibition of odor development and a wide variety of polymers are included in the articles described herein. Following are examples of articles for which it is desirable to reduce microbial growth and/or to inhibit odor development. Microbial growth may be reduced in or on the article. In addition, listed below are examples of end uses in which these articles may be employed.

Articles made antimicrobial and/or odor inhibiting by the methods described herein include packaging for food, personal care (health and hygiene) items, and cosmetics. By “packaging” is meant either an entire package or a component of a package. Examples of packaging components include, but are not limited, to packaging film, liners, absorbent pads packaging, shrink bags, shrink wrap, trays, tray/container assemblies, caps, adhesives, lids, and applicators. Such absorbent pads, shrink bags, shrink wrap, and trays are particularly useful for packaging meat, poultry, and fish, where they prevent the production and concentration of unpleasant odors within the package.

The package may be in any form appropriate for the particular application, such as a can, box, bottle, jar, bag, cosmetics package, or closed-ended tube. The packaging may be fashioned by any means known in the art, such as by extrusion, coextrusion, thermoforming, injection molding, lamination, or blow molding.

Examples of packaging include, but are not limited to, bottles, bottle tips used as applicators of liquid, caps of bottles containing prescription and non-prescription capsules and pills; containers for solutions, creams, lotions, powders, shampoos, conditioners, deodorants, antiperspirants; containers adapted for direct contact with or into the eye, ear, nose, throat, vagina, urinary tract, rectum, skin, and hair; lip product packaging; and caps for containers.

Examples of applicators include lipstick, chapstick, and gloss; packages and applicators for eye cosmetics, such as mascara, eyeliner, shadow, dusting powder, bath powder, blusher, foundation and creams; and pump dispensers and components thereof. These applicators are used to apply substances onto the various surfaces of the body, and reduction of bacterial growth will be beneficial in such applications.

Other packaging components include drink bottle necks, replaceable caps, non-replaceable caps, and dispensing systems; food and beverage delivery systems; baby bottle nipples and caps; and pacifiers. When a liquid, solution or suspension is to be dispensed, the packaging may be fashioned for pipetting individual drops, or spraying a jet or mass of droplets, dispersing fluid under pressure, spreading an emulsion, etc. In addition, packaging identified as inhalers for dispensing pharmaceuticals and other materials having a physiological effect is contemplated.

Besides packaging, end-use, particularly consumer-oriented, applications in which antimicrobial and/or odor inhibiting articles are useful include coatings for food handling and processing apparatus. Such apparatus includes temporary or permanent food preparation surfaces; conveyer belt assemblies and their components; equipment for mixing, grinding, crushing, rolling, pelletizing, and extruding and components thereof; heat exchangers and their components; drains and their components; equipment for transporting water such as, but not limited to, buckets, tanks, pipes, and tubing; and machines for food cutting and slicing and components thereof. A film of a polymer could be treated according to the methods described herein and then heat sealed to the equipment surface. The equipment component may be a screw for mixing and/or conveying that is an element in a single-screw or twin-screw extruder, such as, but not limited to, an extruder used for food processing.

Articles made antimicrobial and/or odor inhibiting by the methods described herein may be used in or as items of apparel, including but not limited to sportswear, activewear, swimwear, intimate apparel, hosiery (such as socks, stockings, pantyhose, legwarmers, and tights), child's garments, medical garments (such as a gown, mask, glove, slipper, bootie, or head covering), athletic uniforms and protective gear (such as protective sports pads, shin guards, and undergarments that regulate heat and moisture transfer) and inserts and liners for such items of apparel (for example, a woven or nonwoven liner or insert for a shoe, boot, or slipper or a liner for a pair of slacks, or underarm shields for a garment). Such garments, inserts, and liners particularly benefit from the inhibition of odor development.

Articles made antimicrobial and/or odor inhibiting by the methods described herein may also be used in or as medical materials, devices, or implants, such as bandages, adhesives, gauze strips, gauze pads, a component of a cast, medical or surgical drapes, syringe holders, catheters, sutures, IV tubing, IV bags, stents, guide wires, prostheses, orthopedic pins, dental materials, pacemakers, heart valves, artificial hearts, knee and hip joint implants, bone cements, vascular grafts, urinary catheter ostomy ports, orthopedic fixtures, pacemaker leads, defibrillator leads, ear canal shunts, cosmetic implants, ENT (ear, nose, throat) implants, staples, implantable pumps, hernia patches, plates, screws, blood bags, external blood pumps, fluid administration systems, heart-lung machines, dialysis equipment, artificial skin, ventricular assist devices, hearing aids, and dental implants.

In the personal hygiene area, articles made antimicrobial and/or odor inhibiting by the methods described herein include personal hygiene articles such as incontinence pads and garments, diapers, training pants, diaper pails, panty liners, sanitary napkins, tampons, and tampon applicators. Such articles particularly benefit from the inhibition of odor development provided by the methods described herein.

Articles made antimicrobial and/or odor inhibiting by the methods described herein also include health care materials such as antimicrobial wipes, baby wipes, personal cleansing wipes, cosmetic wipes, diapers, medicated wipes or pads (for example, medicated wipes or pads that contain an antibiotic, a medication to treat acne, a medication to treat hemorrhoids, an anti-itch medication, an anti-inflammatory medication, or an antiseptic).

Other such articles also include items intended for oral contact, such as a baby bottle nipple, pacifier, apparatus for teeth straightening and accompanying paraphenalia, denture material, cup, drinking glass, toothbrush, or teething toy. In addition, also contemplated are items for children, such as baby books, plastic scissors, toys, and containers of cleaning wipes.

Household articles made antimicrobial and/or odor inhibiting by the methods described herein include telephones and cellular phones, fiberfill, bedding (e.g., mattresses, mattress covers, bedspreads, blankets, bed sheets, pillows, and pillow cases), window treatments, carpet, flooring components, foam padding such as mat and rug backings, upholstery components (including foam padding), nonwoven dryer sheets, laundry softener containing sheets, automotive wipes, household cleaning wipes, counter wipes, shower curtains, shower curtain liners, towels, washcloths, dust cloths, mops, table cloths, refrigerator components, refrigerator surfaces, walls, and counter surfaces. Refrigerator interiors and articles that are used damp or in a damp environment like a bathroom (for example, counter wipes, shower curtains, shower curtain liners, towels, washcloths, and mops) particularly benefit from the inhibition of odor development provided by the present invention.

Articles made antimicrobial and/or odor inhibiting by methods described herein may also include air and water filters that, because of this functionality, can reduce or prevent biofilm growth on the surface of selective separation membranes, for example, ultrafiltration, and microfiltration membranes.

Devices used in fluid transportation and/or storage, such as pipes and tanks, may also benefit from antimicrobial, and/or odor inhibiting polymeric material. A film of polymer made antimicrobial and/or odor inhibiting by the methods described herein may be heat sealed or otherwise affixed to any relevant surface of a pipe or tank to create an anti-fouling surface.

The above listed articles and their components may be made antimicrobial and/or odor inhibiting by the methods described above at any appropriate time before, during or after article manufacture. For example, in making an antimicrobial shower curtain, polymeric material may be treated according to the methods described herein, followed by fashioning a shower curtain from the treated material. Alternatively, the chitosan treatment may be performed after manufacture of the shower curtain. The antimicrobial/odor inhibiting properties of the polymeric material are not believe to be affected significantly by the processes of fashioning the article in its final form.

### EXAMPLES

The present invention is further defined in the following Examples, in which all parts and percentages are by weight and degrees are Celsius. It should be understood that these Examples, while indicating certain embodiments of the invention, are given by way of illustration only. From the above discussion and these Examples, one skilled in the art can ascertain the essential characteristics of this invention, and, without departing from the spirit and scope thereof, can make changes and modifications to adapt the invention to various usages and conditions.

Materials

The chitosan used in the Examples is commercially available under the registered trademark ChitoClear® from Primex Corporation (Siglufjordur, Iceland). The chitosan was used as purchased. It was derived from shrimp shell and had a degree of N-deacetylation of greater than 85%, a molecular weight in the range of about 60,000-200,000 Daltons, and low heavy metal content (less than 20 ppm). Typical particle size of the chitosan was between 60-100 mesh.

Itaconic acid and itaconic anhydride were obtained from Aldrich Chemical Company (Milwaukee, Wis.).

Tetratex® 1303 Expanded PTFE Membrane, 0.45 micron pore rating, 3.5 mils (89 microns) thick was obtained from Donaldson Membranes Group, Donaldson Company, Inc. (Minneapolis, Minn.)

DuPont™ Tyvek® brand Spunbonded Olefin sheet was obtained from E. I. du Pont de Nemours & Co., Inc. (Wilmington, Del.).

Polypropylene film and Mylar® PET film were obtained from DuPont Teijin Films™, Hopewell, Va.

Wood pulp (WP)/PET nonwoven fabric (55% pine WP/45% PET, 2 oz/yd² [68 g/m²]), with a PET side and a WP side was obtained from E. I. du Pont de Nemours & Co., Inc. (Wilmington, Del.).

Nylon 6,6 fabric was obtained from Beacon Fabric & Notions, Lakeland, Fla.

Surface Treatment Method

Substrate sheets were treated using the following procedure:
Pretreatment: plasma, oxygen/argon mixture, 2 KW
Coating: outside surface with itaconic acid/crosslinking triacrylate
Feed rate: 10 g/min
Curing: electron beam, 9 KV, 100 mA

The uncoated substrate sheet was attached to a chilled, rotating drum in a vacuum chamber (300 fpm). The chamber was pumped down to 10⁻⁴ torr, while heating up the evaporator and nozzle. The drum was kept at about −18° C. and rotated in front of the monomer nozzle and the electron gun. After reaching the operating conditions (evaporator temperature 600° F. (316° C.), nozzle temperature 500° F. (260° C.), vacuum 2×10⁴ torr), the plasma treater (for surface cleaning) and E-gun (electron beam source) were turned on, and monomer blend was injected. At the end of the run, everything was turned off, and the chamber was vented and opened. The whole down cycle (load, unload, pumping down and heating up) was 10-15 minutes.

Where the substrate was to be treated with chitosan, the substrate deposited with itaconic acid as described above was then passed through a tray of deionized water and two trays containing 1% chitosan solution in 0.5% aqueous acetic acid. The residence time of the treated substrate in each tray was about 30 sec. The substrate was then dried in a hot air driven oven kept at about 110° C. The drying process was repeated twice to ensure complete drying.

Antimicrobial Test Method

Treated articles were tested for antimicrobial properties by the Shake Flask Test for Antimicrobial Testing of Materials using the following procedure:

1. Inoculate a single, isolated colony from a bacterial or yeast agar plate culture in 15-25 mL of Trypticase Soy Broth (TSB) in a sterile flask. Incubate at 25-37° C. (use optimal growth temperature for specific microbe) for 16-24 h with or without shaking (select appropriate aeration of specific strain). For filamentous fungi, prepare sporulating cultures on agar plates.

2. Dilute the overnight bacterial or yeast culture into sterile phosphate buffer (see below) at pH 6.0 to 7.0 to obtain approximately 10⁵ colony forming units per mL (cfu/mL). The total volume of phosphate buffer needed will be 50 mL×number of test flasks (including controls). For filamentous fungi, prepare spore suspensions at 10⁵ spores/mL. Spore suspensions are prepared by gently resuspending spores from an agar plate culture that has been flooded with sterile saline or phosphate buffer. To obtain initial inoculum counts, plate final dilutions (prepared in phosphate buffer) of 10⁻⁴ and 10⁻³ onto Trypticase Soy Agar (TSA) plates in duplicate. Incubate plates at 25-37° C. overnight.

3. Transfer 50 mL of inoculated phosphate buffer into each sterile test flask containing 0.5 g of material to be tested. Also, prepare control flasks of inoculated phosphate buffer and uninoculated phosphate buffer with no test materials.

4. Place all flasks on a wrist-action shaker and incubate with vigorous shaking at room temperature. Sample all flasks periodically and plate appropriate dilutions onto TSA plates. Incubate at 25 to 37° C. for 16 to 48 h and count colonies.

5. Report colony counts as the number of Colony Forming Units per mL (cfu/mL).

6. The Δt value may be calculated as follows: Δt=C−B, where Δt is the activity constant for contact time t, C is the mean log₁₀ density of microbes in flasks of untreated control materials after X hours of incubation, and B is the mean log₁₀ density of microbes in flasks of treated materials after X hours of incubation. At is typically calculated at 4, 6, or 24 hours and may be expressed as Δtₓ.

Stock Phosphate Buffer:

| | |
|---|---|
| Monobasic Potassium Phosphate: | 22.4 g |
| Dibasic Potassium Phosphate: | 56.0 g |
| Deionized Water: | Bring up volume to 1000 mL |

Adjust the pH of the phosphate buffer to pH 6.0 to 7.0 with either NaOH or HCl, filter, sterilize, and store at 4° C. until use. The working phosphate buffer is prepared by diluting 1 mL of stock phosphate buffer in 800 mL of sterile deionized water.

### Example 1

### Preparation of Itaconic Acid-Grafted, Chitosan-Treated DuPont™ Tyvek® Brand Spunbonded Olefin

Tyvek® brand spunbonded olefin sheets were deposited with itaconic acid as described above. A sample of the treated material was set aside. The rest was treated with a chitosan solution as described above. Chitosan-treated and control spunbonded olefin sheets were then assayed for antimicrobial activity versus E. coli ATCC # 25922 and Listeria monocytogenes Scott A. Results are presented in Table 1.

TABLE 1

| Sample | 0 h | 6 h | 24 h |
|---|---|---|---|
| | E. coli ATCC # 25922, cfu/mL | | |
| Inoculated buffer control | 1.40E+05 | 1.00E+05 | 1.50E+05 |
| Spunbonded olefin sheet treated | 1.40E+05 | 1.75E+04 | 1.40E+05 |
| w. itaconic acid | | | |
| Spunbonded olefin sheet treated | 1.40E+05 | 4.90E+01 | 1.00E+00 |
| w. itaconic acid and chitosan | | | |
| | Listeria monocytogenes Scott | | |
| | A, cfu/mL | | |
| Inoculated buffer control | 8.00E+05 | 5.20E+05 | 5.20E+05 |
| Spunbonded olefin sheet treated | 8.00E+05 | 9.50E+05 | 2.15E+04 |
| w. itaconic acid | | | |
| Spunbonded olefin sheet treated | 8.00E+05 | 4.90E+01 | 1.00E+00 |
| w. itaconic acid and chitosan | | | |

### Example 2

### Preparation of Itaconic Acid-Grafted, Chitosan-Treated Wood Pulp/Polyester Nonwoven Fabric

Wood pulp (WP)/polyester (PET) nonwoven fabric (55% pine WP/45% PET, 2 oz/yd²) sheets were deposited with itaconic acid as described above. A sample of the treated material was set aside. The rest was treated with a chitosan solution as described above. Chitosan-treated and control wood pulp/polyester nonwoven fabric sheets were then assayed for antimicrobial activity versus E. coli ATCC # 25922 and Listeria monocytogenes Scott A. Results are presented in Table 2.

TABLE 2

| Sample | 0 h | 6 h | 24 h |
|---|---|---|---|
| | E. coli ATCC # 25922, cfu/mL | | |
| Inoculated buffer control | 1.40E+05 | 1.00E+05 | 1.50E+05 |
| Wood pulp/polyester sheet treated | 1.40E+05 | 4.75E+04 | 6.00E+04 |
| w. itaconic acid | | | |
| Wood pulp/polyester sheet treated | 1.40E+05 | 4.90E+01 | 1.00E+00 |
| w. itaconic acid and chitosan | | | |
| | Listeria monocytogenes Scott | | |
| | A, cfu/mL | | |
| Inoculated buffer control | 8.00E+05 | 5.20E+05 | 5.20E+05 |
| Wood pulp/polyester sheet treated | 8.00E+05 | 3.55E+05 | 7.00E+04 |
| w. itaconic acid | | | |
| Wood pulp/polyester sheet treated | 8.00E+05 | 2.35E+03 | 1.00E+00 |
| w. itaconic acid and chitosan | | | |

### Example 3

### Preparation of Itaconic Acid-Grafted, Chitosan-Treated Polypropylene Film

Polypropylene film was deposited with itaconic acid as described above. A sample of the treated material was set aside. The rest was treated with a chitosan solution as described above. Chitosan-treated and control polypropylene sheets were then assayed for antimicrobial activity versus E. coli ATCC # 25922 and Listeria monocytogenes Scott A. Results are presented in Table 3.

TABLE 3

| Sample | 0 h | 6 h | 24 h |
|---|---|---|---|
| | E. coli ATCC # 25922, cfu/mL | | |
| Inoculated buffer control | 1.40E+05 | 1.00E+05 | 1.50E+05 |
| Polypropylene film treated | 1.40E+05 | 2.15E+04 | 2.00E+04 |
| w. itaconic acid | | | |
| Polypropylene film treated | 1.40E+05 | 4.90E+01 | 1.00E+00 |
| w. itaconic acid and chitosan | | | |
| | Listeria monocytogenes Scott | | |
| | A, cfu/mL | | |
| Inoculated buffer control | 8.00E+05 | 5.20E+05 | 5.20E+05 |
| Polypropylene film treated | 8.00E+05 | 4.15E+05 | 6.00E+03 |
| w. itaconic acid | | | |
| Polypropylene film treated | 8.00E+05 | 4.90E+01 | 1.00E+00 |
| w. itaconic acid and chitosan | | | |

### Example 4

### Preparation of Itaconic Anhydride-Grafted, Chitosan-Treated Mylar® Polyester Film

Sheets of Mylar® polyester (PET) film were treated as described above, except that itaconic anhydride was used as the graft monomer in place of itaconic acid. A sample of the treated material was set aside. The rest was treated with a chitosan solution as described above. Chitosan-treated and control polyester sheets were then assayed for antimicrobial activity versus E. coli ATCC # 25922 and Listeria monocytogenes Scott A. Results are presented in Table 4.

TABLE 4

| Sample | 0 h | 6 h | 24 h |
|---|---|---|---|
| | E. coli ATCC # 25922, cfu/mL | | |
| Inoculated buffer control | 1.40E+05 | 1.00E+05 | 1.50E+05 |
| Polyester film treated w. itaconic | 1.40E+05 | 6.55E+04 | 1.90E+03 |
| anhydride | | | |
| Polyester film treated w. itaconic | 1.40E+05 | 4.90E+01 | 1.00E+00 |
| anhydride and chitosan | | | |
| | Listeria monocytogenes Scott | | |
| | A, cfu/mL | | |
| Inoculated buffer control | 8.00E+05 | 5.20E+05 | 5.20E+05 |
| Polyester film treated w. itaconic | 8.00E+05 | 4.20E+03 | 1.90E+03 |
| anhydride | | | |
| Polyester film treated w. itaconic | 8.00E+05 | 1.00E+00 | 1.00E+00 |
| anhydride and chitosan | | | |

### Example 5

### Preparation of Itaconic Anhydride-Grafted, Chitosan-Treated Expanded Poly(Tetrafluoroethylene) Film [“ePTFE”]

Sheets of Tetratex® 1303 Expanded PTFE Membrane were treated as in Example 4, with itaconic anhydride as the graft monomer in place of itaconic acid. A sample of the treated material was set aside. The rest was treated with a chitosan solution as described above. Chitosan-treated and control ePTFE sheets were then assayed for antimicrobial activity versus E. coli ATCC # 25922 and Listeria monocytogenes Scott A. Results are presented in Table 5.

TABLE 5

| Sample | 0 h | 6 h | 24 h |
|---|---|---|---|
| | E. coli ATCC # 25922, cfu/mL | | |
| Inoculated buffer control | 1.40E+05 | 1.00E+05 | 1.50E+05 |
| ePTFE film treated w. itaconic | 1.40E+05 | 3.55E+04 | 1.10E+04 |
| anhydride | | | |
| ePTFE film treated w. itaconic | 1.40E+05 | 4.90E+01 | 1.00E+00 |
| anhydride and chitosan | | | |
| | Listeria monocytogenes Scott | | |
| | A, cfu/mL | | |
| Inoculated buffer control | 8.00E+05 | 1.00E+05 | 1.50E+05 |
| ePTFE film treated w. itaconic | 8.00E+05 | 3.55E+04 | 1.10E+04 |
| anhydride | | | |
| ePTFE film treated w. itaconic | 8.00E+05 | 4.90E+01 | 1.00E+00 |
| anhydride and chitosan | | | |

### Example 6

### Preparation of Itaconic Acid-Grafted, Chitosan-Treated Nylon

Nylon 6,6 fabric were deposited with itaconic acid as described above. A sample of the treated material was set aside. The rest was treated with a chitosan solution as described above. Chitosan-treated and control polypropylene sheets were then assayed for antimicrobial activity versus E. coli ATCC # 25922 and Listeria monocytogenes Scott A. Results are presented in Table 6.

TABLE 6

| Sample | 0 h | 6 h | 24 h | Comment |
|---|---|---|---|---|
| | E. coli ATCC # 25922, cfu/mL | | | |
| Inoculated buffer | 1.40E+05 | 1.00E+05 | 1.50E+05 | |
| control | | | | |
| Nylon fabric treated | 1.40E+05 | 8.50E+04 | 2.90E+05 | Slightly |
| w. itaconic acid | | | | turbid |
| | | | | solution |
| Nylon fabric treated | 1.40E+05 | 4.90E+01 | 3.00E+00 | No |
| w. itaconic acid and | | | | turbidity |
| chitosan | | | | |
| | Listeria monocytogenes Scott | | | |
| | A, cfu/mL | | | |
| Inoculated buffer | 8.00E+05 | 5.20E+05 | 5.20E+05 | |
| control | | | | |
| Nylon fabric treated | 8.00E+05 | 6.55E+05 | 2.10E+03 | Slightly |
| w. itaconic acid | | | | turbid |
| | | | | solution |
| Nylon fabric treated | 8.00E+05 | 4.90E+01 | 1.00E+00 | No |
| w. itaconic acid and | | | | turbidity |
| chitosan | | | | |

### Example 7

### Antiodor Efficacy of Itaconic Acid-Grafted, Chitosan-Treated Nylon

Nylon 6,6 (commercially available under the trademarks Supplex® and Tactel® from Invista, Wichita, Kans.) is treated with itaconic acid and chitosan as described in Example 1. Samples are tested for antiodor efficacy at the Monell Chemical Senses Center (Philadelphia, Pa.) as described below.

Methods

Subjects. 20 heterosexual volunteers (10 of each gender) are recruited to participate in organoleptic evaluations of swatches of material that are in contact with the human body for about 14-24 hours. These swatches are attached to cleaned (in non-fragrance detergent) t-shirts (see below) and footwear. Subjects are 18-60 years of age, in good health (self-report), non-smokers, with functioning olfaction and not using steroidal birth-control.

Additionally, 12 male volunteers are recruited to be odor donors. These individuals wear t-shirts and footwear into which symmetrically attached fabric swatches (chitosan-treated and untreated control samples) are sewn. Swatches are labeled to keep the experimental team blind with respect to treatment condition and are sewn into t-shirts in each underarm area, and in the left and right abdominal region, and under the entire foot within the footwear.
The following inclusion criteria are used to recruit odor donors:
   males only (too difficult to control for changes in female body odor over the menstrual cycle)
   18-60 years old (>60 typically changes body odor)
   in good health, by self-report
   willing to go without antiperspirant, underarm deodorant and personal fragrance for the duration of the study
The following exclusion criteria are used in recruiting odor donors:
   diabetics receiving insulin are excluded because of potential changes in body odor
The following inclusion criteria are used to recruit sensory panelists:
   males and females
   18-60 years old (>60 typically reduces odor perception)
   able to breathe through both nostrils
   able to smell and without a history of smell-related problems
   willing to withhold use of personal fragrance on the day of testing
The following exclusion criteria are used in recruiting sensory panelists:
   active cold or allergy at the time of testing
   use of personal fragrance on the day of the test
   females on steroidal birth control
   anosmic or hyposmic

Procedure—Odor donors. Fabric swatches are sewn into identity-coded, cleaned, t-shirts and foot pads in footwear. One of each symmetric pair of swatches in the t-shirt and footwear is made from fabric that is treated with chitosan. The other is untreated and acts as its control. Swatches are coded, e.g., A1L/A2R, or A2L/A1R, where A indicates body location, 1 vs. 2 are treatment conditions, and L vs. R is left/right position. The investigators are blind to the treatment condition until data analyses are completed. T-shirts are worn for 24 hours and footwear (without socks) for 12-14 hours. After receiving the t-shirts and footwear in the early morning, donors perform a light exercise, viz, jogging up and down 5 flights of stairs three times. This is done to generate a light sweat.

Prior to collecting body odors, each donor undergoes a 7-day wash-out phase during which no fragrance or underarm deodorants are worn, and showers/baths are with fragrance-free soap and shampoo. On the day prior to collecting odors and throughout odor collection, volunteers are instructed to restrict their intake of certain foods, e.g., garlic.

Upon completing the odor-collection phase, subjects remove the t-shirts and footpads and place them into labeled plastic zip-lock bags and deliver them to the Monell Center. Upon arrival at Monell, t-shirts and footpads are stored at about −80° C. until their use.

Stimulus samples are prepared by first cutting swatches into thirds and combining pieces from 3 of the males. Care is taken to insure that the treated and control swatches are appropriately matched: treated and control swatches from the same 3 individuals are combined to form one “male stimulus”; however, there are multiple “male stimuli” in each test session. The remaining samples form “donors.” Hence, for each body location, there are 4 “male stimuli” treated with an antimicrobial agent and the 4 analogous control stimuli. Thus, for each body location, sensory panelists evaluate 8 samples.

Sensory Panelists. During a single session lasting 15-25 minutes, sensory panelists are trained in the procedures that are used in the experiment proper. Ratings on a labeled magnitude scale (“LMS,” a psychophysical procedure to assess perceived intensity of a stimulus [Green B. G., Dalton, P., Cowart, B., Shaffer, G., Rankin. K., and Higgins. J. Evaluating the ‘Labeled Magnitude Scale’ for measuring sensations of taste and smell. Chemical Senses, 1996, 21, 323-334]) and ratings of odor pleasantness (on a 23-point scale were −11=Extremely Unpleasant, 0=Neither Pleasant nor Unpleasant, +11=Extremely Pleasant) are provided for the standard odorants phenylethyl alcohol (PEA) and butyric acid (BA) at two concentrations, viz., 10% and 0.1% v/v. Furthermore, in this training session, subjects also make preference choices for stimulus pairs (all possible pairs of PEA vs. BA) and rate the strength of preference on a scale of 0-10. All data collection is performed on a computer that is programmed to prompt the subject for the appropriate responses.

In each of three sessions, subjects evaluate the underarm swatches, the abdominal swatches, or the footwear swatches. Presentation of the three stimulus types is counterbalanced across subjects. Test sessions are at least one day apart.

In a single session, panelists first provide forced-choice preferences in a two-alternative task viz., chitosan treated versus untreated swatches from the same body location from 4 “stimulus males.” Presentation of “stimulus males” is counterbalanced but is repeated such that each “stimulus male” pair is presented 11 times, for a total of 44 forced-choice preferences. For each of the preferences, subjects also provide an estimate of the strength of the preference on the 0-10 scale, with 10 being a very strong preference.

After the forced-choice preferences, subjects are given each “stimulus male” sample (there are 8 samples, the chitosan-treated and control swatches from each of 4 “stimulus males”) and the two control samples and are asked to rate the intensity (using the LMS) and pleasantness and choose a descriptor from a list of 16 (see Table 1). Each of the 10 samples is evaluated 3 times.

TABLE 1
List of descriptors from which odor panelists choose
the most appropriate for the odor being evaluated.

| | | | |
|---|---|---|---|
| Antiseptic | Bad breath | Burnt | Cheese |
| Fishy | Musky | Nothing | Perfume |
| Spoiled milk | Sweaty | Woody | Onion |
| Zoo (lion/tiger | Sulfurous | Floral | Musty (damp |
| cage) | | | basement) |

Results

Preference. Preference data are initially evaluated with a repeated measures analysis of covariance with gender of the panelist as a between groups factor, odor sample as the repeated factor and age as the covariate. Neither age nor gender figure significantly into the results. For underarm, footwear, and abdominal samples, panelists more frequently choose the chitosan-treated samples.

Individual choices are evaluated for significant preference one way or the other. For 44 trials, choosing one sample 29 times or more is a significant (p<0.05) individual preference. For the underarm, footwear, and abdominal samples, many subjects significantly choose the chitosan-treated samples and none significantly choose the controls.

Another way to evaluate preference is to determine a person's choice in the first trial (this becomes important for odors to which people quickly adapt). In the initial trials for each of the odor sources, subjects choose the chitosan-treated underarm, footwear, and abdominal samples more frequently than the untreated samples.

Strength of preference corroborates actual preference. For tests of underarm, footwear, and abdominal samples, in which subjects significantly choose those that are treated with chitosan, the “confidence” of choice, as reflected in the strength of preference, is significantly elevated when individuals choose the chitosan-treated samples relative to the untreated samples.

Perceived Intensity. The three intensity ratings for each sample are averaged and then are analyzed in a repeated measures analysis of covariance with gender as a between groups factor, age as the covariate and repeated measures over the 4 “stimulus males.” The control samples are not included in these analyses.

Analyses of variance of the average intensity rating for the samples from the body are compared with the blank samples, both chitosan-treated and untreated.

Underarm: In a direct comparison, a chitosan-treated sample is rated as significantly less intense than an untreated sample.

Abdomen: Because this region of the body does not produce extensive malodor, there may be no significant difference in perceived intensity between a chitosan-treated sample and an untreated sample.

Feet: In a direct comparison, a chitosan-treated sample is rated as significantly less intense than an untreated sample.

Perceived Pleasantness. The three pleasantness ratings for each sample are averaged and then are analyzed in a repeated measures analysis of covariance with gender as a between groups factor, age as the covariate and repeated measures over the 4 “stimulus males.” The control samples are not included in these analyses.

Analyses of variance of the average pleasantness rating for the samples from the body are compared with the blank samples for chitosan-treated and untreated.

Underarm: In a direct comparison, an untreated sample is rated as significantly more unpleasant than a chitosan-treated sample.

Abdomen: Because this region of the body does not produce extensive malodor, there may be no significant difference in rated pleasantness between a chitosan-treated sample and an untreated sample.

Feet: In a direct comparison, an untreated sample is rated as significantly more unpleasant than a chitosan-treated sample.

Odor Quality. For each stimulus, a descriptor is solicited from each subject by asking subjects to choose an adjective from a list that is provided (Table 1). Each stimulus is presented 3 times. For both chitosan-treated and untreated samples that contact body regions, there are 4 samples each. Hence, across all subjects there are 3×4×20=240 adjective choices. Unexposed samples also are presented 3 times across the 20 subjects resulting in 60 adjective choices. For presentation, results are converted to percent of total adjectives.

Underarm: The adjectives musky, nothing, and sweaty are chosen by the subjects for greater than about 5% of the samples. The adjective sweaty is chosen the most; however, the choice of sweaty is reduced in chitosan-treated samples. The choice of nothing is increased in chitosan-treated samples.

Abdomen: The adjectives nothing, perfume, sweaty, woody, zoo, floral, and musty are chosen by the subjects for greater than about 5% of the samples. The adjective nothing is chosen the most for chitosan-treated and untreated samples.

Feet: The adjectives cheese, musky, nothing, spoiled milk, sweaty, woody, and musty are chosen by the subjects for greater than about 5% of the samples. The adjective sweaty is chosen the most; however, the choice of sweaty is reduced in chitosan-treated samples. The choice of nothing is increased in chitosan-treated samples.

As demonstrated above, chitosan-treated materials reduce malodors associated with regions of the body known to produce significant amounts of odor, viz., the underarms, feet, and abdomen. This results in a preference for swatches that are treated with chitosan over untreated materials when malodors from the body are present.

The positive effects of chitosan are noted in forced-choice preference tests, in evaluations of perceived intensity and pleasantness, and in choices of odor quality (adjective). For underarm, footwear, and abdominal samples, subjects significantly more often choose chitosan-treated swatches over untreated, odorized samples, and rate them as less intense and less unpleasant if they are chitosan-treated. In summary, chitosan significantly reduces malodors originating from the human body.

What is claimed is:

1) A process for making polymeric material antimicrobial and odor inhibiting, the process comprising the steps of:
a) mixing a graft monomer with a crosslinking agent to produce a blend;
b) feeding the blend into a hot evaporator under vacuum;
c) flash evaporating the blend through a nozzle;
d) recondensing the blend onto polymeric material;
e) exposing the recondensed blend to ultraviolet or electron beam radiation, whereby the recondensed blend and the polymeric material react to form a crosslinked graft copolymer;
f) contacting the crosslinked graft copolymer with a solution comprising a chitosan agent selected from the group consisting of chitosan, chitosan salts, chitosan-metal complexes, and chitosan derivatives;
g) optionally, contacting the crosslinked graft copolymer with a solution containing a metal salt; and
h) drying the crosslinked graft copolymer;
wherein the contacted crosslinked graft copolymer is antimicrobial and odor inhibiting.

2. The process of claim 1, wherein the polymeric material comprises at least one polymer selected from the group consisting of naturally occurring polymers; cotton; wood; flax; shellac; silk; wool; natural rubber; leather; combinations of naturally occurring polymers; synthetic polymers; homopolymers; mixtures, blends, and copolymers of polyesters; polyetheresters; polyethers; polyamides; polyimides; polyetherimides; polyacetals; polystyrene; polyphenylene oxide; polyphenylene sulfide; polysulfones; poly(meth)acrylates; liquid crystalline polymers; polyetherketones; fluorine-containing polymers; acrylonitrile-styrene-butadiene resins; styrene-butadiene block copolymers; polycarbonates; cellulose-based polymers; urea formaldehyde resins; polyacrylonitrile; epoxy resins; polyurethanes; melamine-formaldehyde resins; silicones; butyl rubber; polychloroprene; and polyolefins.

3. The process of claim 2, wherein the polyolefin is a homopolymer of ethylene; a homopolymer of propylene; a copolymer derived from ethylene and one or more C₃-C₈ alpha-olefins; a copolymer derived from propylene and one or more C₄-C₈ alpha-olefins; polypropylene selected from atactic polypropylene; isotactic polypropylene; syndiotactic polypropylene; biaxially oriented polypropylene (BOPP); metallocene-catalyzed polypropylene; polyethylene selected from high density polyethylene, low density polyethylene, linear low density polyethylene, metallocene catalyzed polyethylene, very low density polyethylene, ultrahigh molecular weight polyethylene, and high performance polyethylene; copolymers of ethylene and propylene; copolymers derived from a combination of ethylene and at least one monomer selected from methyl acrylate, ethyl acrylate, n-butyl acrylate, methyl methacrylate, acrylic acid, methacrylic acid, and carbon monoxide; copolymers derived from a combination of propylene and at least one monomer selected from methyl acrylate, ethyl acrylate, n-butyl acrylate, methyl methacrylate, acrylic acid, methacrylic acid, and carbon monoxide; copolymers of olefins with a diolefin, wherein the olefins are selected from ethylene, propylene, and ethylene with other olefins; copolymers of ethylene and tetrafluoroethylene.

4. The process of claim 3, wherein the diolefin is selected from linear aliphatic nonconjugated dienes of at least six carbon atoms, norbornadiene, dicyclopentadiene, ethylidene norbornene, and butadiene.

5. The process of claim 1, wherein the graft monomer is selected from a group consisting of a thermally stable unsaturated monomer containing amine-reactive functional groups; methacrylic acid; acrylic acid; glycidyl methacrylate; 2-hydroxy ethylacrylate; 2-hydroxy ethyl methacrylate; beta-carboxylethyl acrylate; beta-carboxyethyl methacrylate; diethyl maleate; monoethyl maleate; di-n-butyl maleate; maleic anhydride; maleic acid; fumaric acid; itaconic acid; itaconic anhydride; dodecenyl succinic anhydride; 5-norbornene-2,3-anhydride; and nadic anhydride (3,6-endomethylene-1,2,3,6-tetrahydrophthalic anhydride).

6. The process of claim 1, wherein the chitosan containing solution comprises 0.1% to 3% by volume of chitosan and further comprises 0.5% to 1.0% by volume of aqueous acetic acid.

7. The process of claim 1, wherein the metal salt is selected from the group consisting of water-soluble zinc salt, water-soluble copper salt, water-soluble silver salt, and mixtures thereof.

8. The process of claim 1, wherein the chitosan has a degree of N-deacetylation of greater than 85%, a molecular weight in the range of from about 60,000 to about 200,000 Daltons, and heavy metal content less than 20 ppm.

9. An article comprising polymeric material made antimicrobial and odor inhibiting by the process of claim 1.

10. The article of claim 9, wherein the polymeric material is selected from the group consisting of naturally occurring polymers; cotton; wood; flax; shellac; silk; wool; natural rubber; leather; combinations of naturally occurring polymers; synthetic polymers; homopolymers; mixtures, blends, and copolymers of polyesters; polyetheresters; polyethers; polyamides; polyimides; polyetherimides; polyacetals; polystyrene; polyphenylene oxide; polyphenylene sulfide; polysulfones; poly(meth)acrylates; liquid crystalline polymers; polyetherketones; fluorine-containing polymers; acrylonitrile-styrene-butadiene resins; styrene-butadiene block copolymers; polycarbonates; cellulose-based polymers; urea formaldehyde resins; polyacrylonitrile; epoxy resins; polyurethanes; melamine-formaldehyde resins; silicones; butyl rubber; polychloroprene; and polyolefins.

11. The article of claim 10, wherein the polyolefin is a homopolymer of ethylene; a homopolymer of propylene; a copolymer derived from ethylene and one or more C₃-C₈ alpha-olefins; a copolymer derived from propylene and one or more C₄-C₈ alpha-olefins; polypropylene selected from atactic polypropylene; isotactic polypropylene; syndiotactic polypropylene; biaxially oriented polypropylene (BOPP); metallocene-catalyzed polypropylene; polyethylene selected from high density polyethylene, low density polyethylene, linear low density polyethylene, metallocene catalyzed polyethylene, very low density polyethylene, ultrahigh molecular weight polyethylene, and high performance polyethylene; copolymers of ethylene and propylene; copolymers derived from a combination of ethylene and at least one monomer selected from methyl acrylate, ethyl acrylate, n-butyl acrylate, methyl methacrylate, acrylic acid, methacrylic acid, and carbon monoxide; copolymers derived from a combination of propylene and at least one monomer selected from methyl acrylate, ethyl acrylate, n-butyl acrylate, methyl methacrylate, acrylic acid, methacrylic acid, and carbon monoxide; copolymers of olefins with a diolefin, wherein the olefins are selected from ethylene, propylene, and ethylene with other olefins; copolymers of ethylene and tetrafluoroethylene.

12. The article of claim 11, wherein the diolefin is selected from linear aliphatic nonconjugated dienes of at least six carbon atoms, norbornadiene, dicyclopentadiene, ethylidene norbornene, and butadiene.

13. The article of claim 9, wherein the graft monomer is selected from a group consisting of a thermally stable unsaturated monomer containing amine-reactive functional groups; thermally stable unsaturated carboxylic anhydride; thermally stable unsaturated dianhydride methacrylic acid; crylic acid; glycidyl methacrylate; 2-hydroxy ethylacrylate; 2-hydroxy ethyl methacrylate; beta-carboxylethyl acrylate; beta-carboxyethyl methacrylate; diethyl maleate; monoethyl maleate; di-n-butyl maleate; maleic anhydride; maleic acid; fumaric acid; itaconic acid; itaconic anhydride; dodecenyl succinic anhydride; 5-norbornene-2,3-anhydride; and nadic anhydride (3,6-endomethylene-1,2,3,6-tetrahydrophthalic anhydride).

14. The article of claim 9, wherein the metal salt is selected from the group consisting of water-soluble zinc salt; water-soluble copper salt; water-soluble silver salt; and mixtures thereof.

15. The article of claim 9 selected from the group consisting of film; a membrane; a laminate; knit fabric; woven fabric; nonwoven fabric; fiber; a filament; yarn; a pellet; coating; foam; a blown article; a solution cast article; a laminated article; an injection molded article; a blow molded article; a thermoformed article; a knit article; a woven article and a spun article.

16. The article of claim 9, wherein the article is selected from the group consisting of packaging; food handling apparatus; food processing apparatus; a food dispensing system; a beverage dispensing system; an ingested article; a dental appliance; a garment; a household article; a health care article; a medical device; a personal grooming article; personal hygiene article; a storage container for fluids; a transportation container for fluids; a filter and a separation membrane.

17. The article of claim 9, wherein the article is selected from the group consisting of
packaging; a package; a container; a bottle; a box; a jar; a can; a bag; a closed-ended tube; a packaging component; a package for food; a package for a beverage; a packaging liner; a lid; a replaceable container cap; a disposable container cap; film used in packaging; packaging for flesh foods; absorbent pads for flesh food packaging; a shrink bag; a food tray; fast food packaging; a soft drink bottle neck; food handling apparatus; food processing apparatus; a food dispensing system; a beverage dispensing system; a conveyor belt assembly; components of a conveyor belt assembly; temporary and permanent food preparation surfaces; equipment for food preparation; heat exchangers; drains; buckets; tanks; pipes; tubing;
an ingested article; a capsule; a pill; a liquid;
an orthodontic appliance; a component of an orthodontic appliance; denture material; a toothbrush; a teeth cleaning appliance;
clothing; sportswear; activewear; swimwear; underwear; hosiery; socks;
stockings; pantyhose; tights; a legwarmer; a child's garment; a clothing insert; a clothing liner; an underarm shield; a woven or nonwoven liner or insert for footwear; an athletic uniform; athletic protective gear; sports pad; shin guard; undergarment that regulates heat or moisture transfer;
a household article; fiberfill for pillows; bedding; a mattress; a mattress cover; a bedspread; a blanket; a bed sheet; a pillow; a pillow case; window treatments; carpet; a flooring component; an upholstery component; foam padding; an automotive wipe; a nonwoven dryer sheet; a laundry softener-containing sheet; a household cleaning wipe; a counter wipe; a towel; a washcloth; a dust cloth; a mop; a tablecloth; a refrigerator component; a refrigerator surface; a shower curtain; a shower curtain liner; a wall; a counter surface;
a health care article; a bandage; an adhesive; gauze strip; a gauze pad; a cast; medical drape; surgical drape; a medical garment; a hospital gown; a surgical mask; a surgical glove; surgical footwear; surgical head covering; an inhaler; a medical device; a medical implant; a syringe holder; a catheter; a suture; IV tubing; an IV bag; a stent; guide wires; a prosthesis; an orthopedic pin; a dental implant; a pacemaker; a pacemaker lead; a defibrillator lead; a heart valve; an artificial heart; a joint implant; bone cement; a vascular graft; a urinary catheter ostomy port; an orthopedic fixture; an ear canal shunt; a cosmetic implant; an ENT implant; surgical staples; an implantable pump; a hernia patch; a surgical plate; a surgical screw; a blood bag; an external blood pump; fluid administration systems; a heart-lung machine; a dialysis equipment; artificial skin; ventricular assist devices; a hearing aid;
children's articles; a baby bottle; a teething toy; a baby bottle nipple; a pacifier; a child's book; plastic scissors; a toy; a diaper pail; a container for cleansing wipes; a personal cleansing wipe; a baby wipe;
a personal grooming article; cosmetics; a cosmetics package; a cosmetic wipe; lipstick; lip balm; eye shadow; eyeliner; mascara; body powder; bath powder; blusher; face make-up; shampoo; conditioner; deodorant; antiperspirant; body lotion; body cream; face powder; a pump dispenser; a mascara wand; a medicated wipe; a cosmetics brush; a dropper; a dropper tip; a lipstick applicator; an eyeliner applicator; an eye shadow applicator; a liquid; a solution; a suspension;
a personal hygiene article; a diaper; training pants; an incontinence pad; an incontinence garment; a panty liner; a sanitary napkin; a tampon; a tampon applicator;
a separation membrane; an ultrafiltration membrane; a microfiltration membrane;
transportation container for fluids; storage container for fluids;
an air filter; a water filter;
a boat component; boat hull; and boat motor.

18. A process for inhibiting odor in an article, wherein the article comprises polymeric material and is selected from the group consisting of personal hygiene articles, an incontinence pad or garment, a diaper, training pants, a diaper pail, a panty liner, a sanitary napkin, a tampon, a tampon applicator, packaging, flesh food packaging, shrink wrap, a shrink bag, a tray, an absorbent pad, apparel, sportswear, activewear, swimwear, intimate apparel, hosiery, a child's garment, a medical garment, an athletic uniform, athletic protective gear, an insert for apparel, a liner for apparel, underarm shield for a garment, a protective sports pad, a shin guard, an undergarment that regulates heat or moisture transfer, an insert for footwear, a surgical gown, a surgical mask, a surgical glove, medical footwear medical head covering, a sock, a stocking, pantyhose, tights, a legwarmer, household article, counter wipes, shower curtains, shower curtain liners, towels, washcloths, mops,
the process comprising treating the polymeric material according to the process of claim 1.
